(19) United States
(12) Patent Application Publication
(10) Pub. No.: US 2026/0069446 A1
(43) Pub. Date: Mar. 12, 2026

(54) SYSTEM AND DEVICE FOR MAXILLOMANDIBULAR FIXATION

(71) Applicant: Invisian Medical, LLC, Prairie Village, KS (US)

(72) Inventors: Jan Creidenberg, Mission Hills, KS (US); Brogan Moroney, Kansas City, MO (US); Omar Khan, Kansas City, MO (US); Andrew Dirks, Overland Park, KS (US)

(73) Assignee: Invisian Medical, LLC, Prairie Village, KS (US)

(21) Appl. No.: 19/325,002

(22) Filed: Sep. 10, 2025

Related U.S. Application Data
(60) Provisional application No. 63/693,345, filed on Sep. 11, 2024.

(51) Int. Cl. A61F 5/058

(52) U.S. Cl. CPC A61F 5/05891

(57) ABSTRACT

An arch bar system for maxillomandibular fixation is disclosed. A contourable, trim-to-fit bar includes a series of elongated oval apertures oriented substantially parallel to the bar axis. The apertures guide wire, coated suture, or other ligatures at varied approach angles, limit vertical clearance to resist slippage, and cooperate with intervening structural columns to distribute loads while the bar is cinched to teeth. Low-profile depending attachment features—cleats and/or J-hooks—project below the lower edge to receive interarch wire loops, guiding elastics, or other connectors. All patient-facing edges and transitions are radiused to minimize gingival and mucosal trauma. Bars may be metal or engineered polymer and segmentable for partial-arch use. Methods include threading ligatures through successive apertures to secure each bar and joining opposing bars at the depending features. Kits include paired bars, ligatures, interarch connectors, and tools.

### RELATED APPLICATION

This application claims priority from U.S. Provisional application No. 63/693,345 filed on Sep. 11, 2024. The contents of that application are incorporated herein by reference in its entireties.

### FIELD OF THE DISCLOSURE

The present disclosure relates generally to intraoral fixation devices used to stabilize the maxilla and mandible. More particularly, it concerns arch bars and associated ligation/interarch coupling components for achieving maxillomandibular fixation (MMF) in oral and maxillofacial surgery, including management of facial trauma, orthognathic procedures, and temporomandibular joint reconstruction. In certain embodiments, the devices comprise contourable bars with elongated oval apertures for tooth attachment via wire or other ligatures and depending attachment features (e.g., cleats or hooks) for connection between opposing arches.

### BACKGROUND

Maxillomandibular fixation (MMF) is a critical procedure associated with jaw surgery for the treatment of facial trauma, temporomandibular joint (TMJ) total joint replacement surgery, and orthognathic surgery (corrective jaw surgery). MMF is utilized to bring the mandible (lower jaw) into alignment to ensure pre-morbid or desired occlusion following surgery or treatment of non-displaced mandible fractures.

Conventional techniques include tooth-borne wiring systems (e.g., Ivy eyelets, Erich arch bars) and bone-borne systems (e.g., intermaxillary-fixation screws and other bone-supported devices). In current practice, MMF may be used intraoperatively to register occlusion and, in selected cases, postoperatively to maintain reduction.

Historically, external immobilization methods such as the Barton bandage (1819) and Gunning-type vulcanite splints (introduced during the U.S. Civil War) established early paradigms for jaw stabilization, especially in edentulous patients. These approaches evolved toward tooth-borne interdental wiring techniques and laid the groundwork for modern MMF.

Interdental wiring matured in the early 20th century. In 1922, Robert H. Ivy popularized the loop (eyelet) wiring technique, enabling rapid occlusal fixation across a wide range of fracture patterns. These developments standardized the concept of locking the maxilla and mandible together via the dentition.

Mid-century, the Erich arch bar became a widely used, durable method for prolonged MMF. Arch bars remain familiar to surgeons but are associated with lengthy application/removal, difficult oral hygiene, and exposure of staff to sharp wire ends, which can increase the risk of glove perforation and needle-stick injury.

From the 1970s onward, rigid internal fixation (miniplate osteosynthesis) reduced or eliminated the need for extended postoperative MMF in many cases by providing stable open reduction along ideal lines of osteosynthesis. As a result, many workflows now emphasize brief intraoperative occlusal registration and earlier jaw mobilization when fracture stability allows.

To address arch-bar drawbacks, intermaxillary-fixation (IMF) screws were introduced in 1989 as bone-borne anchors for MMF. Literature reports that IMF screws can shorten application time and reduce sharps exposure compared with traditional wiring, while providing reliable occlusal control for temporary or short-term fixation; however, screws can loosen or injure dental roots and are generally favored for limited durations.

More recently, hybrid/bone-supported arch bars have been described, combining dentition-spanning bars with bone fixation to improve efficiency and staff safety while maintaining occlusal control. Randomized and systematic comparisons report broadly similar occlusal outcomes among techniques, with meaningful differences in placement time, glove perforations, and hygiene parameters. Technique selection therefore remains highly operator-dependent and case-specific.

Despite these advances, persistent needs remain. Conventional MMF systems can: (i) require significant chair/operating room time; (ii) expose personnel to sharps; (iii) compromise oral hygiene and patient comfort; (iv) be difficult to apply in poor or mixed dentitions; and (v) integrate inconsistently with workflows that prioritize rigid fixation and early mobilization (e.g., orthognathic surgery or TMJ reconstruction). Accordingly, there remains a need for devices and methods that provide faster, safer, and more hygienic MMF, that maintain reliable occlusal control across varied dentitions and clinical settings, and that integrate seamlessly with modern maxillofacial surgical protocols.

### SUMMARY OF THE INVENTION

The present disclosure relates to an arch bar for maxillomandibular fixation (MMF) and intraoral stabilization. In one aspect, the device comprises an elongate bar contoured to follow the dental arch and carrying a plurality of specially designed oval apertures configured to facilitate secure, durable attachment of the bar to teeth.

The oval geometry provides generous lead-in for wire or other tie materials at multiple angles of approach, promotes uniform load distribution around the opening during tightening, and reduces local stress concentration compared with circular or sharp-cornered holes. The apertures accommodate metallic ligature wire as well as alternative fibrous, monofilament, or polymeric ties, allowing the clinician to select a fixation medium appropriate to dentition, hygiene considerations, and procedural speed.

In another aspect, the bar incorporates a plurality of uniquely designed and spaced hooks that drop below the lower edge of the bar. These depending features present protected engagement points for interarch connection—e.g., intermaxillary wiring, guiding elastics, or other intraoral connectors—while preserving a clear tie path between opposing arches. The position, size, and spacing of the knobs/hooks are selected to (i) provide ample utilization of ties without slippage, (ii) maintain consistent vectoring of interarch forces, and (iii) minimize interference with lip/cheek tissues during function.

All patient-facing edges and surfaces of the bar—including the apertures and the hooks—are engineered to minimize abrasiveness and soft-tissue trauma. In certain embodiments, edges are radiused, chamfered, or otherwise blended; corners at the apertures are rounded; hook tips are bulbous or domed; and tie-receiving channels are recessed or shielded to keep ligature ends away from gingiva and oral mucosa. Optional surface finishes (e.g., polishing, passivation, or coatings) further reduce friction and plaque retention. The low-profile cross-section and smooth transitions are configured to improve patient comfort during the period of fixation and to facilitate oral hygiene.

The disclosed arch bar can be provided in straight or pre-contoured segments and in multiple sizes or curvatures to accommodate maxillary and mandibular arches. In representative implementations, the long axis of each oval aperture is oriented to accept ties in oblique or orthogonal directions relative to the tooth surfaces, permitting secure lashing to crowns, interproximal embrasures, or bonded anchors. The hooks may be arranged in a repeating pitch along the bar, staggered relative to the apertures for strength, and shaped to retain ties under cyclic loading while permitting rapid placement and removal.

The device is preferably manufactured from biocompatible metals (e.g., stainless steel, titanium) or from high-strength polymeric/ceramic composites. Representative fabrication processes include stamping or laser-cutting followed by forming and finishing, metal injection molding, or additive manufacturing to generate the aperture and hook geometries with controlled radii. The device may be supplied sterile as a single-use article or as a reusable instrument with validated reprocessing instructions. Kits may include bars for upper and lower arches, assorted wire gauges and/or non-metallic ties, and tools for contouring, placement, and removal.

In use, the clinician positions the bar along the labial/buccal aspects of the teeth, passes wire or alternative ties through the oval apertures and around selected tooth surfaces or bonded buttons, and tightens to cinch the bar into secure apposition. Opposing upper and lower bars are then interconnected via the depending hooks using wire, guiding elastics, or other intraoral devices to establish and maintain the desired occlusion and interarch force vectors. The smooth, rounded architecture of the bar, apertures, and hooks reduces the likelihood of gingival irritation during the fixation interval and streamlines both application and removal.

Nothing in this summary is intended to limit the scope of the claims; rather, it provides a non-exhaustive overview of certain features and combinations that may be claimed.

It is an object of the disclosed apparatus to provide an arch bar with oval apertures that enhances secure, durable attachment to teeth using wire or alternative tie materials.

It is a further object of the disclosed arch bar to enable multi-material fixation, including metallic ligatures and non-metallic ties, through aperture geometry that supports various tie paths and angles.

It is a further object of the disclosed arch bar to incorporate hooks that drop below the lower edge of the bar to facilitate reliable connection between opposing arches using wire, guiding elastics, or other intraoral devices.

It is a further object of the disclosed arch bar to minimize abrasiveness and soft-tissue trauma via rounded edges, domed hook tips, and smooth, low-profile surfaces.

It is a further object of the disclosed arch bar to improve efficiency and versatility of MMF placement by offering strategically spaced attachment features.

It is a further object of the disclosed arch bar to enhance retention while maintaining easy placement and removal.

It is a further object of the disclosed arch bar to offer manufacturing flexibility (metal or polymer constructions; various forming methods) that supports sterilization and single use.

It is a further object of the disclosed arch bar to provide a system and kit that streamlines clinical workflow, including bars, ties, and application tools configured for rapid, hygienic deployment.

### BRIEF DESCRIPTION OF THE DRAWINGS

FIG. 1 illustrates a perspective view of an embodiment of arch bar;

FIG. 2 illustrates a front elevation view of an embodiment of the arch bar;

FIG. 3 illustrates a side elevation view of an embodiment of the arch bar;

FIG. 4 illustrates a perspective view of a second embodiment of the arch bar;

FIG. 5 illustrates a front elevation view of the second embodiment of the arch bar;

FIG. 6 illustrates a side elevation view of the second embodiment of the arch bar;

FIG. 7 illustrates a perspective view of an embodiment of an arch bar contoured to fit a dental arch;

FIG. 8 illustrates a perspective view of an embodiment of an arch bar in position and contoured to a dental arch;

FIG. 9 illustrates an arch bar being trimmed to a length that allows ligation to as many teeth as possible;

FIG. 10 illustrates the placement of the cleat shaped hooks toward the occlusal surfaces and as symmetrically as possible in the upper and lower jaw;

FIG. 11 illustrates the use of ties as ligatures to affix the arch bar to the dentition;

FIG. 12 illustrates the creation of a circumdental loop around two teeth on the same arch by advancing a needle driver to an embrasure anterior to the first embrasure;

FIG. 13 illustrates an introducer is advanced through the side of a clasp;

FIG. 14 illustrates the application of tension to the suture to eliminate slack on the lingual side of the teeth;

FIG. 15 illustrates the clasp pulled tightly against the arch bar;

FIG. 16 illustrates the application of multiple clasps for affixing the arch bar;

FIG. 17 illustrates the repeating of the process to affix the bar on the opposite arch with the cleats facing the occlusal surfaces;

FIG. 18 illustrates the application of wire loops to the arch bar;

FIG. 19 illustrates the application of guiding elastics to the arch bar;

FIG. 20 illustrates the cutting of the wire loops to release the arch bars from maxillomandibular fixation;

FIG. 21 illustrates the utilization of a wire cutter to sever the polymeric suture loop;

FIG. 22 illustrates a process flow diagram of MMF using the disclosed arch bar and system; and

FIG. 22A illustrates a continuation of the process flow diagram of FIG. 22.

### DETAILED DESCRIPTION

The following description is merely exemplary in nature and does not limit the present teachings, application, or uses. Throughout this specification, reference numerals will be used to refer to like elements. Additionally, the embodiments disclosed below are not intended to be exhaustive or to limit the invention to the precise forms disclosed in the following detailed description. Rather, the embodiments are chosen and described so that others skilled in the art can utilize their teachings.

The drawings furnished are intended to illustrate and plainly disclose presently envisioned embodiments to one of skill in the art but are not intended to be manufacturing level drawings or renditions of final products and may include simplified conceptual views to facilitate understanding or explanation. As well, the relative size and arrangement of the components may differ from that shown and still operate within the spirit of the invention.

As used herein, the word “exemplary” or “illustrative” means “serving as an example, instance, or illustration.” Any implementation described herein as “exemplary” or “illustrative” is not necessarily to be construed as preferred or advantageous over other implementations. All the implementations described below are exemplary implementations provided to enable persons skilled in the art to practice the disclosure and are not intended to limit the scope of the appended claims.

Unless otherwise defined, all technical and scientific terms used herein have the same meaning as commonly understood by one of ordinary skill in the art to which this disclosure belongs. The terminology used herein is for the purpose of describing example embodiments only and is not intended to be limiting. As used herein, the singular forms “a”, “an”, and “the” may be intended to include the plural forms as well, unless the context clearly indicates otherwise. The terms “comprises”, “comprising”, “including”, and “having” are inclusive and therefore specify the presence of stated features, steps, operations, elements, and/or components, but do not preclude the presence or addition of one or more other features, steps, operations, elements, components, and/or groups thereof. The method steps, processes, and operations described herein are not to be construed as necessarily requiring their performance in the order discussed or illustrated, unless specifically identified as an order of performance. It is also to be understood that additional or alternative steps can be employed.

When an element, object, device, module, apparatus, component, region or section, etc., is referred to as being “on”, “engaged to or with”, “connected to or with”, or “coupled to or with” another element, object, device, module, apparatus, component, region or section, etc., it can be directly on, engaged, connected or coupled to or with the other element, object, device, module, apparatus, component, region or section, etc., or intervening elements, objects, devices, modules, apparatuses, components, regions or sections, etc., can be present. In contrast, when an element, object, device, module, apparatus, component, region or section, etc., is referred to as being “directly on”, “directly engaged to”, “directly connected to”, or “directly coupled to” another element, object, device, module, apparatus, component, region or section, etc., there may be no intervening elements, objects, devices, modules, apparatuses, components, regions or sections, etc., present. Other words used to describe the relationship between elements, objects, devices, modules, apparatuses, components, regions, or sections, etc., should be interpreted in a like fashion (e.g., “between” versus “directly between,” “adjacent” versus “directly adjacent,”etc.).

As used herein the phrase “operably connected to” will be understood to mean two or more elements, objects, devices, modules, apparatuses, components, etc., that are directly or indirectly connected to each other in an operational and/or cooperative manner such that operation or function of at least one of the elements, objects, devices, modules, apparatuses, components, etc., imparts or causes operation or function of at least one other of the elements, objects, devices, modules, apparatuses, components, etc. Such imparting or causing of operation or function can be unilateral or bilateral.

As used herein, the term “and/or” includes all combinations of one or more of the associated listed items. For example, A and/or B includes A alone, or B alone, or both A and B. Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Although the terms first, second, third, etc., can be used herein to describe various elements, objects, devices, modules, apparatuses, components, regions, or sections, etc., these elements, objects, devices, modules, apparatuses, components, regions, or sections, etc., should not be limited by these terms. These terms may be used only to distinguish one element, object, device, module, apparatus, component, region, or section, etc., from another element, object, device, module, apparatus, component, region, or section, etc., and do not necessarily imply a sequence or order unless clearly indicated by the context.

Moreover, it will be understood that various directions such as “upper”, “lower”, “bottom”, “top”, “left”, “right”, “first”, “second” and so forth are made only with respect to explanation in conjunction with the drawings, and that components may be oriented differently, for instance, during transportation and manufacturing as well as operation. Because varying and different embodiments may be made within the scope of the concept(s) taught herein, and because many modifications may be made in the embodiments described herein, it is to be understood that the details herein are to be interpreted as illustrative and non-limiting.

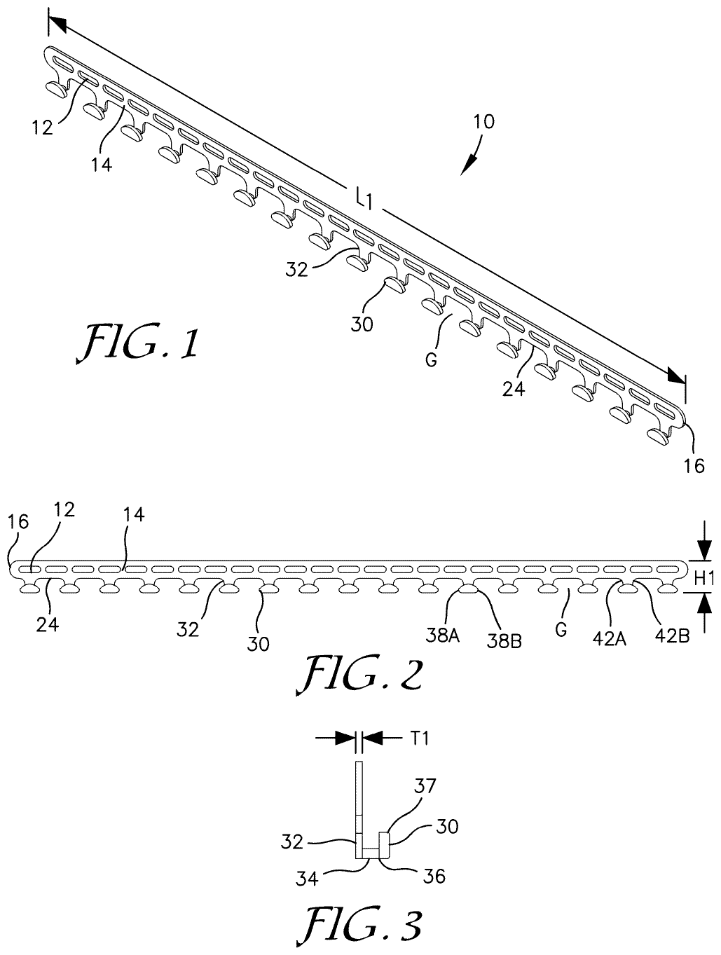

Illustrated at FIG. 1 is a first embodiment of an arch bar 10 specifically designed with length, width and height dimension to be formed to and precisely fitted around and attached to the teeth on the maxillary (upper jaw) arch and mandibular (lower jaw) arch, while allowing ample space between the bars for wire, guiding elastics or other ligature materials to securely connect the upper and lower bars to achieve maxillomandibular fixation.

The arch bar 10 is preferably fabricated with a length L1 of between 5.0 and 5.5 inches and is designed to be cut to any length to be fitted to the unique size and shape of dental arches of each individual patient. The height H1 of the arch bar 10, as illustrated at FIG. 2 is preferably in the range of 0.120 inches to 0.140 inches with a preferred height of 0.130 inches. The thickness T1 of the arch bar 10, as illustrated at FIG. 3 is preferably in the range of 0.014 to 0.018 inches with a preferred thickness of 0.016 inches. The arch bar 10 may be cut into small segments that can be affixed to one tooth or several teeth, depending on patient specific anatomical or other patient characteristics, as well as a range of clinical needs.

As illustrated at FIGS. 1-2, the arch bar 10 features a plurality of elongated apertures (slots) 12 and structural columns 14 designed to allow secure attachment of the arch bar 10 to the teeth while also providing the flexibility of the arch bar 10 to shape to the dental arches and the strength needed to maintain integrity of the arch bar during fixations. The shape, dimensions and spacing of the apertures are designed and optimized to affix the arch bar 10 to the teeth using dental occlusion ties such as those disclosed at U.S. Pat. No. 11,045,235, which are constructed of suture with a round, smooth, non-ribbed plastic coating, a rigid metal needle/introducer on one end and a self-locking, infinitely adjustable locking mechanism on the other end.

The series of oval-shaped apertures 12 are oriented horizontally relative to the arch bar's longitudinal axis 16. These apertures 12 serve as guide channels for the securement ties, allowing them to pass through the arch bar 10 and wrap around the teeth to affix the bar.

The dimensions of each aperture accommodate the securement ties while minimizing vertical clearance, ensuring a snug fit that prevents unwanted movement. The apertures 12 are preferably in the range of 0.150 to 0.175 inches in width (at the widest point) and in the range of 0.040 to 0.060 inches in height with spacing between the apertures 12 in the range of 0.030 to 0.040 inches. Each of the longitudinally opposed ends of the apertures 12 has a radius in the range of about 0.020 inches. Evenly spaced along the bar, the apertures 12 and structural columns 14 provide consistent force distribution and reliable fixation across the dental arch.

The elongated design of the apertures 12 simplifies the application process, allowing for rapid and accurate placement. The shape, dimensions and spacing of the apertures also provide versatility to affix the bar to teeth using wire or ligatures constructed of other material. The columns 14 between the apertures 12 anchor the securement ties. These columns 14 have a width of about 0.035 inches and a thickness of about 0.016 inches are engineered to withstand the forces exerted during securement to the teeth with dental occlusion ties, while their smooth, rounded surfaces prevent damage to the ties and enhance patient comfort.

The seamless integration of the columns 14 with the arch bar 10 ensures overall structural integrity, eliminating potential failure points. In the first embodiment, as illustrated at FIGS. 1-3, the bottom edge 24 of the arch bar 10 includes a plurality of cleats 30, intentionally spaced apart about 0.30 inches (center to center of the cleats) to allow clinicians to attach wire, guiding elastics or other ligature material precisely where needed.

The cleats 30 include a first segment 32 extending downwardly from the arch bar 10 in the range of about 0.080 inches to 0.085 inches. The downwardly extending segment 32 has a thickness in the range of about 0.013 to 0.018 inches and a width of about 0.044 inches. A second segment 34 extends outwardly from the first segment in the range of about 0.040 inches to 0.045 inches and has a thickness in the range of about 0.022 to 0.027 inches. The cleat 30 is located at the distal end 36 of the second segment 34.

The cleat 30 has a thickness of about 0.0.25 inches and the upper edge 37 of the cleat has a radius of about 0.089 inches while the two side edges 38A, 38B of the cleat 30 have a radius of about 0.020 inches. The first segment 32 also has a radius of about 0.045 inches on both sides 42A and 42B where the first segment 32 connects to the arch bar 10. While the cleat 30 as disclosed above has a radiused upper edge 37 the cleat shape may also be spherical, semi-spherical, circular prism-shaped, or crescent-shaped.

The gap G between the side edges 38A, 38B of each cleat 30 is preferably in the range of 0.120 to 0.180 inches with a preferred gap of about 0.150 inches. The cleats 30 being disposed near the bottom of the arch bar 10 facilitate placement of the cleats 30 closer to the occlusal plane, which has been shown to improve the quality of occlusion achieved with maxillomandibular fixation. This embodiment of the arch bar 10 may alternatively include two cleats 30, each engineered for versatility to attach wire, guiding elastics or other ligature materials needed to achieve intermaxillary fixation.

The design of the cleat 30 includes sufficient height and width to prevent wires, guiding elastics or other ligature materials from slipping off. The cleat design further includes smooth or beveled edges to minimizes tissue damage, patient discomfort and potential damage to guiding elastics. The cleat 30 includes a relatively large surface area distal to the arch bar 10, which helps to prevent tissue irritation and patient discomfort.

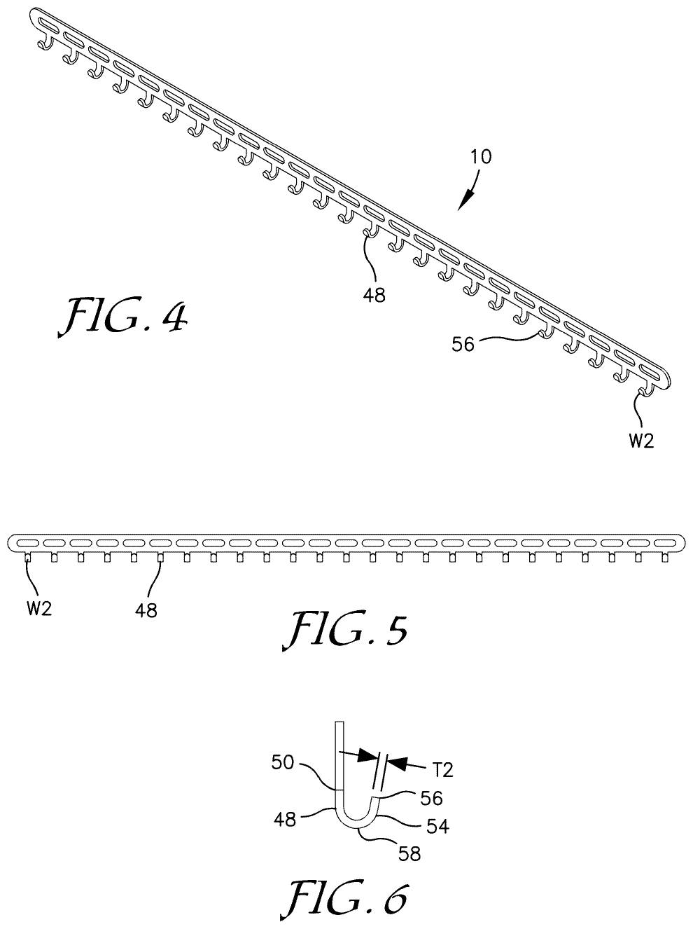

As illustrated at FIGS. 4-6, a second embodiment for attachment points is one also fabricated from the same configuration of the arch bar 10 as detailed above to include the aperture 12 and column 14 configuration. In this second embodiment, referred to as the J-hook, a hook segment 48 extends downward from the lower edge 50 of the arch bar 10 and then curves away from the arch bar 10, creating an attachment point for wire, guiding elastics or ligatures of other materials.

As illustrated at FIGS. 4-5, the width W2 of the entire hook segment 48 is about 0.040 inches, while the thickness T2 of the hook segment 48 is in the range of 0.013 inches to 0.019 inches. As illustrated at FIG. 6, the hook segment 48 extends downwardly from the arch bar line of departure 50 a total of about 0.074 inches. The hook segment 48 includes an upwardly extending segment 54 that terminates at a radiused edge 56. The upwardly extending segment 54 commences at a curved portion 58 with the bend in the curved portion having a radius of about 0.025 inches and terminates at the radiused edge 56. The edge 56 preferably has a radius of about 0.020 inches.

The curved portion 58, radiused edge 56 and upwardly extending segment 54 jointly facilitate the secure attachment of wires, guiding elastics or other ligature materials. This second embodiment's smooth, curved design, and smooth or beveled edges reduce the risk of irritation to mucosal tissues, patient discomfort or damage to guiding elastics. The J-hook embodiment is low profile based on the tight radius of the curved portion 58 ensuring unobstructed access during procedures, reduced risk of tissue damage and improved patient comfort.

The arch bar 10 is preferably fabricated from high-strength, biocompatible materials, such as titanium and alloys of the metal, stainless steel or non-metal materials, such as engineered plastics, offering the required strength, flexibility, durability and reliability required for maxillomandibular fixation.

The method of using the arch bar 10 is discussed in greater detail below, however, a summary of the procedure is to position the arch bar 10 along either the maxillary or mandibular dental arch, and the securement ties or other ligatures are threaded through the apertures, wrapped around the teeth, and passed through a second aperture. As the ties or other ligatures are tightened, the bar is secured to the teeth securely and durably without excessive movement. Once both the upper and lower bars are secured, the hooks are connected using wire, elastics or other ligature material, achieving the necessary maxillomandibular fixation. This configuration provides rigidity for jaw stabilization during healing, while allowing for controlled elastic movements when required.

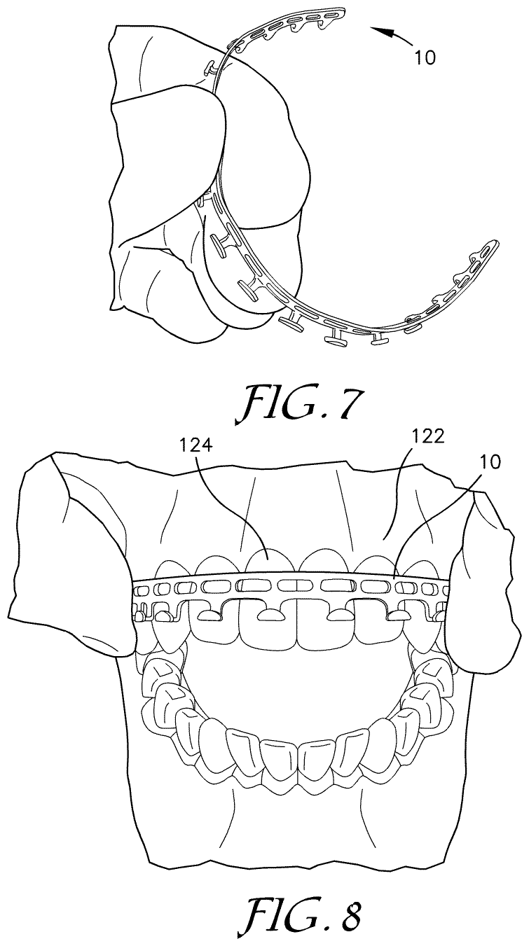

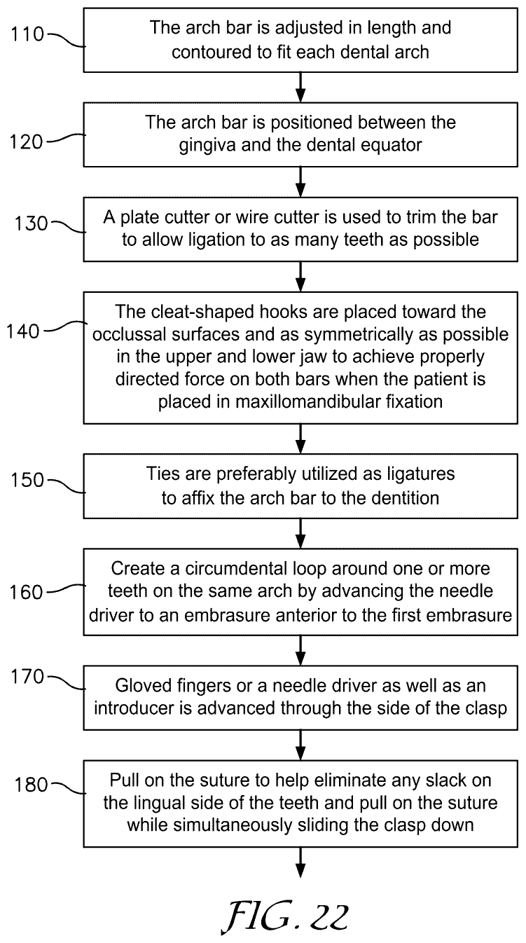

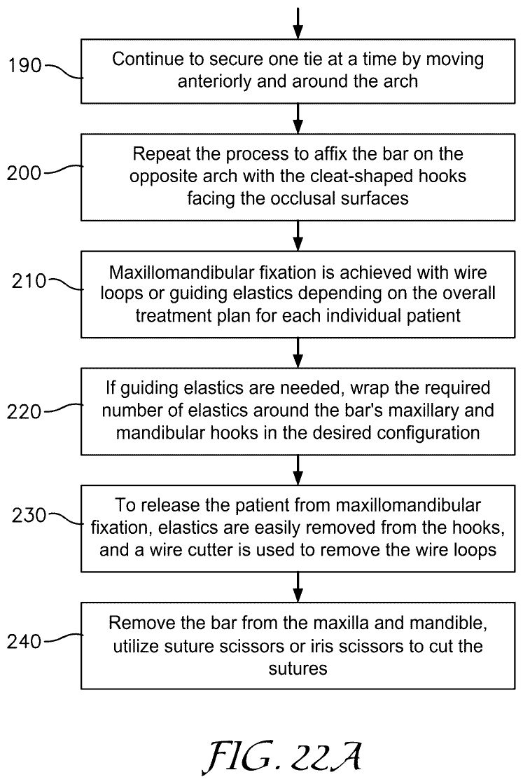

A more expansive discussion of the method of utilization of the arch bar 10 begins with FIG. 7, the arch bar 10 is provided in one length (approximately 5 inches/128 mm) and must be adjusted in length and contoured to fit each dental arch as set forth at step 110 at FIG. 22. A bar 10 that is too short may not provide adequate fixation. As illustrated at FIG. 8, and at step 120, the arch bar 10 must be positioned between the gingiva 122 and the dental equator, in other words on the dentition 124 just below the gingiva.

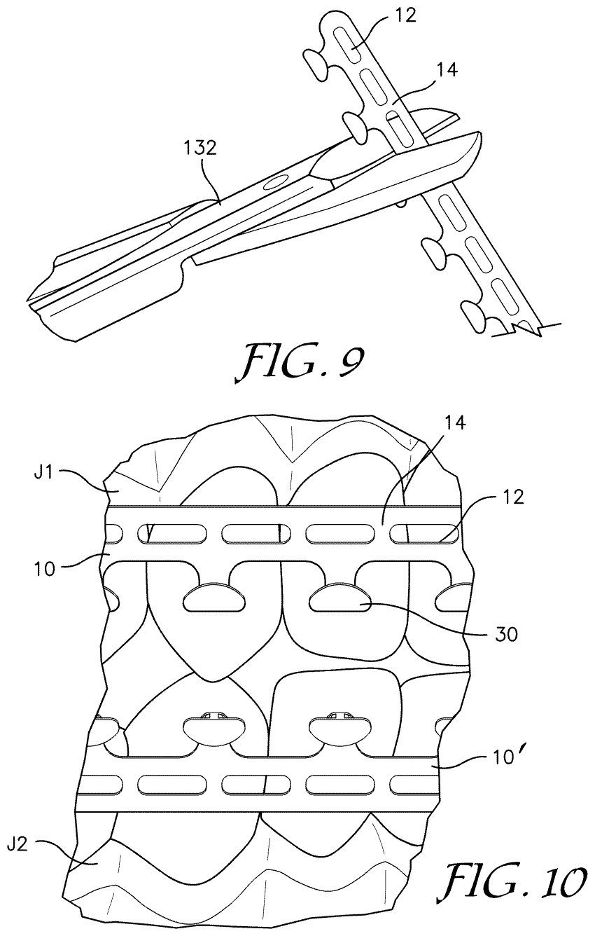

As illustrated at FIG. 9, and at step 130, a plate cutter or wire cutter 132 is used to trim the bar 10 to allow ligation (circumdental attachment) to as many teeth 124 as possible. The bar 10 should not extend past the most distal tooth or protrude into the gingiva. It is recommended to trim the bar 10 as close as possible to a column 14 between apertures 12. As illustrated at FIG. 10, and at step 140, the hook segments 48 and cleat 30 are placed toward the occlusal surfaces and as symmetrically as possible in the upper J1 and lower jaw J2 to achieve properly directed forced on both bars 10, 10′ when the patient is placed in maxillomandibular fixation.

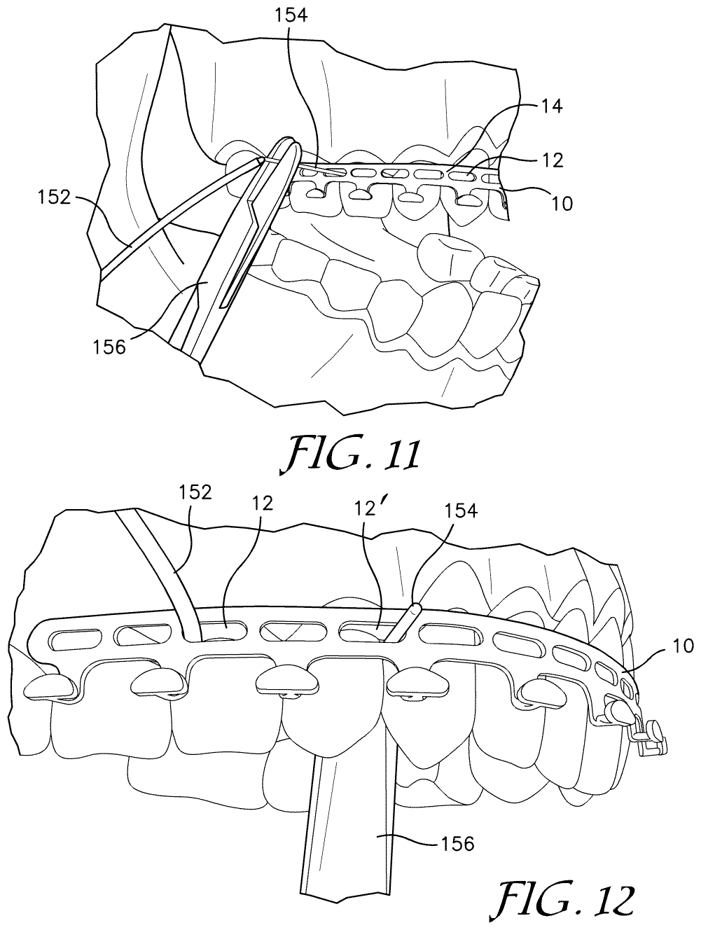

As illustrated at FIG. 11, and at step 150, ties 152 are preferably utilized as ligatures to affix the arch bar 10 to the dentition. The affixation begins posteriorly in the molars or premolars. The tie introducer 154 is inserted through an aperture 12 that aligns with an apical embrasure (valley between teeth) of a molar or pre-molar. The introducer 154 is used to displace the interdental gingival papilla and push through the embrasure from buccal to lingual. The introducer 154 is grasped with a needle driver 156 on the lingual side, pulling the suture until the clasp is 2.5-5.0 cm from the teeth.

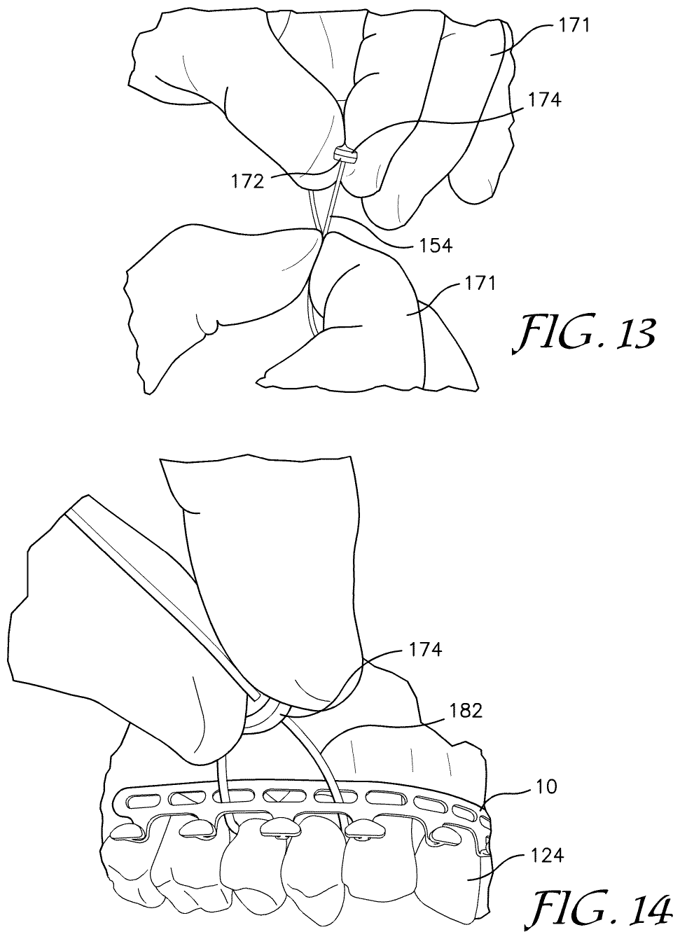

As illustrated at FIG. 12, and at step 160, the next step is to create a circumdental loop around one or more teeth on the same arch 10 by advancing the needle driver 156 to an embrasure anterior to the first embrasure. Push through the embrasure to exit on the buccal side and then push the introducer 154 through a different aperture 12′ on the bar 10 anterior to the first aperture 12. The next step is illustrated at FIG. 13, and at step 170, gloved fingers 171, or optionally, a needle driver 156 are used to advance the introducer 154 through the designated side 172 of the clasp 174.

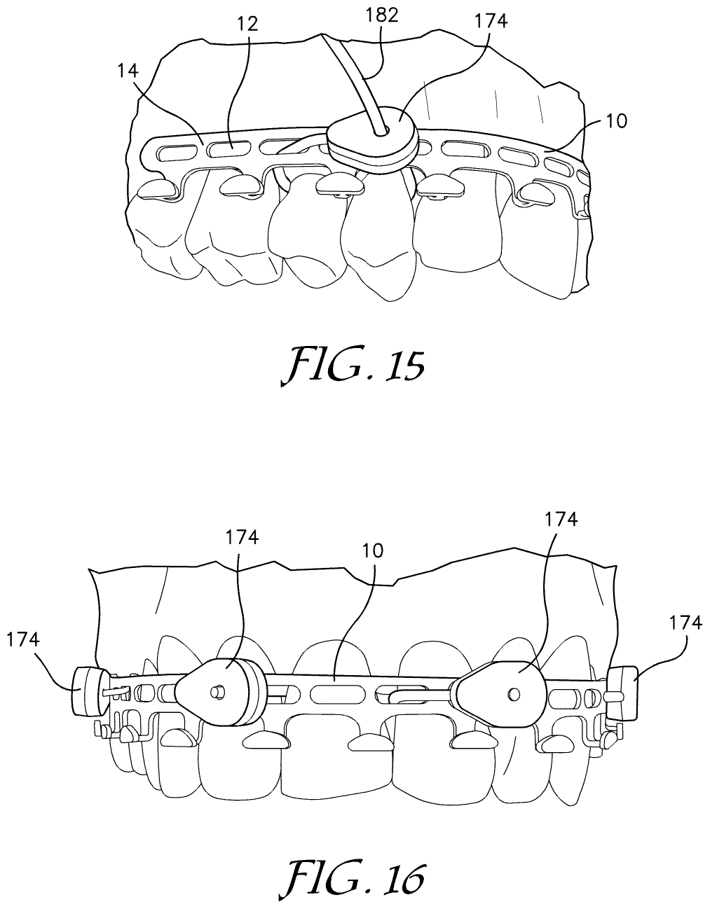

Next, as illustrated at FIGS. 14 and 15, and at step 180 the user pulls on the polymer coated suture, or dental occlusion tie 182 to eliminate any slack on the lingual side of the teeth and pulls on the suture while simultaneously sliding the clasp 174 down. The user continues to tighten until the clasp 174 is secured against the bar 10 and the bar is snugly secured to the teeth 124. As illustrated at FIG. 16, and at step 190, the user continues to secure one tie at a time by moving anteriorly and around the arch. This process is preferably repeated with at least two additional ties (for a minimum total of 3 ties per arch). It is up to the professional judgment of the user to determine the optimal number of ties needed to securely affix the bar to the dentition to achieve stable maxillomandibular fixation.

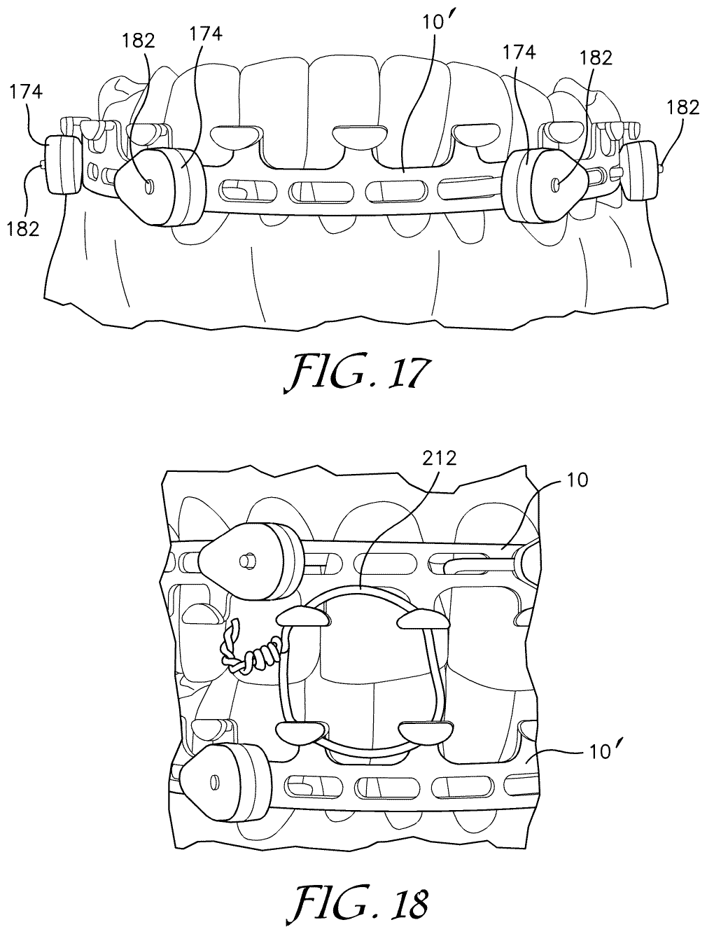

As illustrated at FIG. 17, and at step 200, the user repeats the process to affix the bar on the opposite arch 10′ with the cleat-shaped hooks facing the occlusal surfaces. Cut the suture 182 flush with the clasp or leave a 1-2 cm “tail” if further tightening intraoperative is required. If leaving the bars 10, 10′ on post-operatively for a period of days or weeks, a blade is preferably employed to sever the suture flush with the clasp, resulting in greater comfort for the patient.

As illustrated at FIG. 18, and at step 210, maxillomandibular fixation is achieved with wire loops 212 or guiding elastics depending on the overall treatment plan for each individual patient. For rigid maxillomandibular fixation, a wire loop 212 is placed over the bar's maxillary and mandibular J-hooks 48 or cleats 30 and twisted to tighten. The hooks can be accessed from the top or either side, providing good access even if a clasp 174 is near the J-hook 48 or cleat 30. A minimum of three wires are recommended to provide stable fixation (a posterior wire loop on each side, along with an anterior loop).

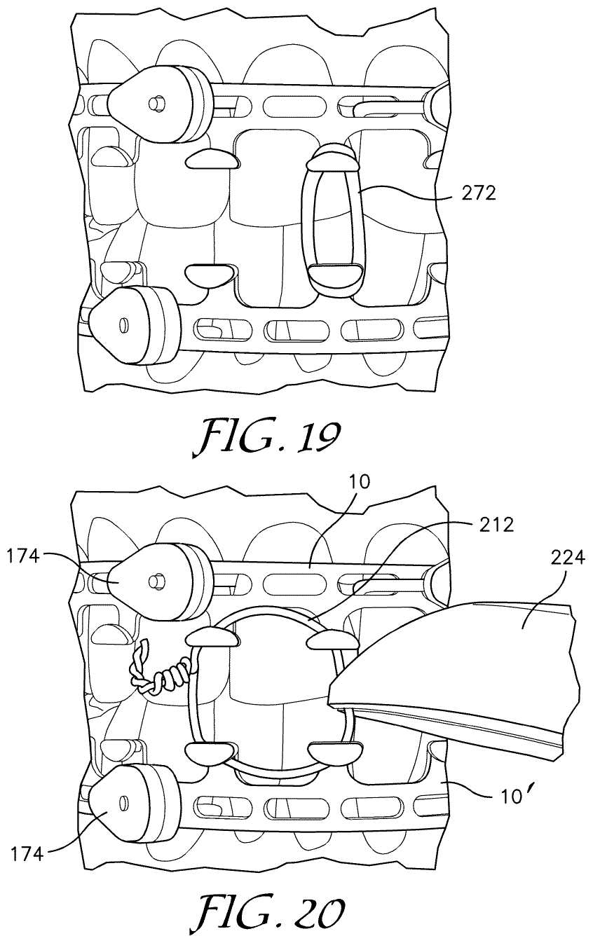

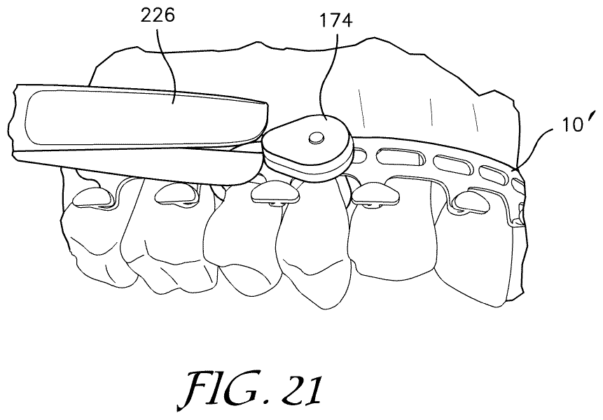

As illustrated at FIG. 19, and at step 220, if guiding elastics 222 are needed, wrap the required number of elastics around the bar's maxillary and mandibular hooks 48 or cleats 30 in the desired configuration. The hooks or cleats can be accessed from the top or either side. To release the patient from maxillomandibular fixation, as illustrated at FIG. 20, and at step 230, elastics 222 are easily removed from the hooks or cleats, and a wire cutter 224 is used to remove the wire loops 212. To remove the bar 10, 10′ from the maxilla and mandible, utilize suture scissors or iris scissors 226 to cut the sutures as illustrated at FIG. 21 and at step 240. FIGS. 22-22A illustrate a flow diagram of the process of MMF using the disclosed arch bars 10, 10′ and system.

In view of the many possible embodiments to which the principles of the disclosed invention may be applied, it should be recognized that the illustrated embodiments are only examples of the disclosure and should not be taken as limiting the scope of the invention. Rather, the scope of the invention is defined by the following claims. We therefore claim as our invention all that comes within the scope of these claims.

The disclosure presented herein is believed to encompass at least one distinct invention with independent utility. While the at least one invention has been disclosed in exemplary forms, the specific embodiments thereof as described and illustrated herein are not to be considered in a limiting sense, as numerous variations are possible. Equivalent changes, modifications, and variations of the variety of embodiments, materials, compositions, and methods may be made within the scope of the present disclosure, achieving substantially similar results. The subject matter of the disclosed arch bar and system includes all novel and non-obvious combinations and sub-combinations of the various elements, features, functions and/or properties disclosed herein and their equivalents.

Benefits, other advantages, and solutions to problems have been described herein regarding specific embodiments. However, the benefits, advantages, solutions to problems, and any element or combination of elements that may cause any benefits, advantage, or solution to occur or become more pronounced are not to be considered as critical, required, or essential features or elements of any or all the claims of at least one invention.

Many changes and modifications within the scope of the instant disclosure may be made without departing from the spirit thereof, and the one or more inventions described herein include all such modifications. Corresponding structures, materials, acts, and equivalents of all elements in the claims are intended to include any structure, material, or acts for performing the functions in combination with other claim elements as specifically recited. The scope of the one or more inventions should be determined by the appended claims and their legal equivalents, rather than by the examples set forth herein.

Benefits, other advantages, and solutions to problems have been described herein regarding specific embodiments. Furthermore, the connecting lines, if any, shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the inventions.

In the detailed description herein, references to “one embodiment,” “an embodiment,” “an example embodiment,” etc., indicate that the embodiment described may include a feature, structure, or characteristic, but every embodiment may not necessarily include the feature, structure, or characteristic. Moreover, such phrases are not necessarily referring to the same embodiment. Further, when a feature, structure, or characteristic is described relating to an embodiment, it is submitted that it is within the knowledge of one skilled in the art to affect such feature, structure, or characteristic relating to other embodiments whether explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S. C. § 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

The method steps described in this disclosure are not intended to be limited to the specific order presented in the claims. While the steps may be described sequentially for clarity, it is understood that the order of the steps may be altered or rearranged without departing from the scope of the invention. The method is intended to be flexible in its application, allowing for variations in the sequence of steps based on specific implementation requirements, processing capabilities, or other considerations. As such, the invention encompasses all orders of execution for the steps, provided the desired result or function of the invention is achieved.

The disclosed system and method have been described above with reference to one or more preferred embodiments, it will be appreciated that various changes or modifications may be made without departing from the scope of the invention as defined in the appended claims.

We claim:

1. An arch bar for intraoral fixation, comprising:
a generally elongate body configured to be contoured to a dental arch; and
a plurality of elongated, oval or substantially oval apertures formed through the body and oriented with their major axes generally parallel to a longitudinal axis of the body, wherein
the oval-aperture geometry is configured to receive and guide flexible ligature material at multiple angles of approach and to distribute fixation loads while limiting vertical clearance to inhibit migration of the ligature relative to the body, and
wherein edges defining the apertures are rounded to reduce contact abrasion with gingival and oral mucosal tissues.

2. The arch bar of claim 1, further comprising structural columns between adjacent apertures, the columns maintaining bar integrity during tightening of the ligature material.

3. The arch bar of claim 1, wherein each aperture exhibits an aspect ratio (major axis to minor axis) of about 2.5:1 to about 6:1 and opposed end regions of the aperture include continuous radii to reduce stress concentration.

4. The arch bar of claim 1, wherein the body has a height of about 0.120-0.140 in. and a thickness of about 0.012-0.020 in. and is trim-to-fit in length or segmentable for placement on one or more teeth.

5. The arch bar of claim 1, further comprising depending attachment features selected from cleats and hooks that extend below a lower edge of the body to provide interarch connection points for wire, guiding elastics, or other intraoral connectors, the depending attachment features comprising at least one J-hook or at least one cleat; the J-hook including a straight stem that extends about 0.040-0.090 in. below the lower edge of the body and terminating in a return portion subtending about 180-240° with an inner radius of about 0.020-0.030 in. and a throat opening of about 0.030-0.055 in. to admit an elastic or wire; the cleat including a pair of opposed arms defining a slot width of about 0.035-0.045 in. and an undercut saddle depth of about 0.015-0.030 in., with a projection below the lower edge of about 0.070-0.10 in. ; all external edges and corners of the J-hook and cleat being rounded with a radius of about 0.015-0.025 in., each feature presenting a labial/oral mucosa-facing surface area of about 0.005-0.020 in².

6. A method of affixing an arch bar to teeth, comprising:
providing an arch bar including a plurality of elongated, oval or substantially oval apertures oriented with major axes generally parallel to a longitudinal axis of the arch bar;
contouring and trimming the arch bar to fit along a dental arch;
threading a flexible ligature through a first one of the oval apertures, passing the ligature through an interdental embrasure, and returning the ligature through a second oval aperture at a different location; and
tightening the ligature to draw the arch bar into secure apposition with the teeth, wherein the oval-aperture geometry guides the ligature and maintains a low-profile, controlled tie path that resists vertical slippage.

7. The method of claim 6, wherein the ligature comprises wire, coated suture, polymeric filament, or a combination thereof, optionally including a rigid introducer and/or a self-locking clasp.

8. The method of claim 6, further comprising repeating the threading and tightening at multiple sites along the arch to create distributed fixation, and, when an opposing arch bar is present, coupling the arch bars together using wire loops or guiding elastics engaged to depending attachment features of at least one of the bars.

9. The method of claim 6, wherein threading begins posteriorly and proceeds anteriorly around the arch, with at least three ligatures placed per arch.

10. The method of claim 6, further comprising releasing the patient from interarch fixation by removing elastics and/or severing wire loops and removing the arch bar by cutting the ligatures flush with the bar.

11. A system for intraoral fixation, comprising:
(a) at least one arch bar;
(b) a plurality of ligatures dimensioned to pass through oval apertures of the arch bar and around one or more teeth; and
(c) tools for placement and removal, wherein the oval-aperture geometry of the arch bar cooperates with the ligatures to permit rapid threading at variable angles and stable, low-profile fixation along the dental arch.

12. The system of claim 11, further comprising a pair of arch bars sized for maxillary and mandibular arches and a plurality of interarch connectors selected from wire loops and guiding elastics.

13. The system of claim 11, wherein the arch bar or bars are fabricated from a biocompatible metal or engineered polymer, include rounded or polished surfaces, and are supplied sterile as single-use or reusable components with validated reprocessing instructions.

14. The system of claim 11, wherein the oval apertures are evenly spaced along the arch bar with inter-aperture spacing of about 0.030-0.040 in. and end radii of about 0.020 in., and the structural columns between adjacent apertures have a width of about 0.035 in. to anchor ligatures without cutting.

15. The system of claim 11, wherein the system further includes pre-contoured bars and straight trim-to-fit bars in lengths of about 5.0-5.5 in., and ligatures comprising wire and non-metallic ties to enable clinician-selected fixation modalities.

16. A method of achieving maxillomandibular fixation, comprising:
positioning an arch bar along a dental arch between gingiva and the dental equator;
trimming and contouring the arch bar to fit the arch;
threading a securement tie through a first oval aperture of the arch bar, passing the securement tie through an interdental embrasure to the lingual side, looping the securement tie around one or more teeth, and returning the securement tie through a second oval aperture;
tightening the securement tie to cinch the arch bar against the teeth to obtain a secure, low-profile fixation at multiple sites along the arch;
repeating the foregoing steps for an opposing arch; and
coupling the opposing arch bars together at depending attachment features using at least one of:
a wire loop, a guiding elastic, or another intraoral connector, thereby establishing maxillomandibular fixation.

17. An arch bar for maxillomandibular fixation, comprising:
an elongate bar configured to be contoured to a patient's maxillary or mandibular dental arch and to be trimmed in length;
a plurality of oval, elongated apertures formed through the bar and oriented generally horizontally relative to a longitudinal axis of the bar, the apertures dimensioned to receive a securement tie and to guide the securement tie around one or more teeth to affix the bar to the dentition;
a plurality of structural columns defined between adjacent apertures to maintain bar integrity under fixation loads; and
a plurality of depending attachment features that extend below a lower edge of the bar and are positioned and shaped to couple to an opposing arch bar using a wire, an elastic, or another intraoral connector, wherein edges of the bar, the apertures, and the depending attachment features are rounded or beveled to minimize abrasiveness and soft-tissue trauma to gingival and oral mucosal tissues.

18. A system for establishing maxillomandibular fixation, comprising:
(a) a pair of arch bars sized for the maxillary and mandibular arches;
(b) a plurality of securement ties each configured to be threaded through oval apertures of the arch bars and around teeth to affix the arch bars to the dentition;
(c) a plurality of interarch connectors selected from wires and guiding elastics configured to connect the pair of arch bars at the depending attachment features, and
(d) placement tools including at least one of a plate or wire cutter, needle driver, and scissors; wherein the oval apertures, structural columns, and depending attachment features of the arch bars cooperate to provide secure attachment to teeth and reliable interarch coupling while minimizing abrasiveness to gingival and oral mucosal tissues.
